(19) United States
(12) Patent Application Publication
(10) Pub. No.: US 2018/0031512 A1
(43) Pub. Date: Feb. 1, 2018

(54) A Disposable Pesticide Analysis Kit Not Containing Enzymes, DNA or Components for Land Type Pesticide Analysis

(71) Applicant: Betül Yasemin Akar, Izmir (TR)

(72) Inventor: Betül Yasemin Akar, Izmir (TR)

(21) Appl. No.: 15/550,471

(22) PCT Filed: Mar. 9, 2016
(86) PCT No.: PCT/TR2016/000032

§ 371 Date: Aug. 11, 2017

(30) Foreign Application Priority Data
Feb. 13, 2015 (TR) 2015/01788

(51) Int. Cl. G01N 27/30; G01N 27/327

(52) U.S. Cl. CPC G01N 27/308; G01N 27/3275; G01N 33/00

(57) ABSTRACT

This invention, land-type pesticide analysis methods in electro chemical pesticide residue analysis in order to make an analysis of samples of water and plant extracts for disposable enzyme, does not contain live microorganisms, DNA and components residue analysis Kit and the kit to be used with the analysis methods.

### BACKGROUND OF THE INVENTION

### Field of Invention

This invention concerns a residue analysis kit set and the analysis methods to be used with this kit that does not include enzymes, live microorganisms, DNA or its components for the taking samples from plant sap and the analysis of the samples for the purpose of conducting pesticide residue analysis in a field type analysis device with electrochemical methods.

### Related Art

Pesticide analysis is currently conducted in laboratory conditions. The sample extracted by standard sampling are put through the preliminary processes necessary for the analysis, they are broken down, ground up, subjected to chemical solutions, put through a centrifugal process and filtered before being placed in the device for analysis. Each of these processes requires separate equipment and separate amounts of time and each process in conducted separately.

If pesticide analysis is conducted electrochemically in laboratories with potentiostat devices, the sample is filtered by processing with chemical solutions, put through a centrifugal process and then distilled with electrodes covered with special material. The electrode is connected to the device and the chemical process can be monitored and converted into data by way of the device.

The devices on the market have a variety of different properties but their use is simple and can be easily connected to any potentiostat by way of a connection cable or a connector. These electrodes are independent pieces of equipment that can be used at the sample analysis stage; the processes of the taking of the sample and the putting the sample through preliminary procedures are conducted separately in different locations.

Furthermore, in conjunction with developing technology, there are single use pesticide analysis kits that can be used for pesticide analysis in soil, foodstuffs in all environments. The AgroScreen Ticket kits produced by the company Neogen, the Organophosphate/Carbamate Assay Kit sets produced by the company Abraxis and RaPID Assay Organophosphate Carbamate Screen Kit sets belonging to Strategic Diagnostic Inc., that are among the companies that produce these kits, have been produced based on the principle of determining the biochemical reaction of pesticides containing organophosphate and carbamate with the cholinesterase enzyme acetylcholine and they illustrate the ensuing reactions by way of their own activation solutions to produce a visible change in colour. The kits are biological sensors with the purpose of showing enzyme activity; their operating areas are limited to pesticide active materials in the group of pesticides containing organophosphates and carbamate.

There also exists kits produced by different companies that use butyrylcholine that determine the reaction between the active materials of the group of pesticides containing organophosphates and carbamate and butyrylcholine, which is also one of the cholinesterase enzymes, by way of bio-activators and shows the reaction on the kit in the form of a change in colour.

These kits do not differentiate between the active materials of pesticides containing organophosphates or carbamate during analysis; they only show the presence of this group of pesticides and it does not determine the amount. A change in colour occurs within a few minutes on the biochemical surface of the kit after the sample is prepared as per the instructions in the instruction manual and applied on the kit. The presence of the pesticide group containing organophosphates and/or carbamate active materials is indicated by the hue of the colour.

The diagnostic value limit of these kits intended for use in the field produces by the listed companies and other companies is very high. For example, the lowest value of the determination of Aldicarb, one of the active materials of the group of pesticides containing organophosphates or carbamate, in the AgriScreen Ticket kits produced by the company Neogen is 0.2 ppm. The legal MRL limit for this pesticide active material is 0.01-0.02 ppm. The sale and export of agricultural products containing more Aldicarb that the limit is prohibited. Likewise, the company's kit has a determination value for the active material Carbaryl that is over 7.0 ppm. The legal MRL limit of the active material Carbaryl is 0.1 ppm. The lower limit for the pesticide active material phorate in the company kit is 3.0 ppm; the legal MRL upper limit for this active material is 0.05 ppm. The lower limit for the pesticide active material methamidophos in the company kit is 4.0 ppm; the legal MRL upper limit is 0.01 ppm. The values are similar in kits produced by other companies.

These kits that show the biological reaction between acetylcholine, butyrylcholine and similar enzymes with pesticide active materials within the plant are useful for users but as the diagnostic value lower limit is much higher than legal values, the areas of application are limited and they can only be used for informative purposes. The kits in question are also unable to determine the existence of pesticide metabolites.

Organophosphate and carbamate compounds are only two of the pesticide active material groups still used in agriculture. Compounds with organophosphate, compounds with organochlorine, compounds with carbamate, the pyretrin group of compounds, halogen and oxygens, amine and hydrazine derivatives, dinitrofenol and dinitrofenol esters, compounds containing sulphur, organic stannous compounds, compounds containing copper, dithiocarbamates, phthalimides, nitro compounds, pyrimidines, triazoles, triazines, benzimidazoles, chloric aliphatic acids, dinitroamin analines, carbamide compounds, uracils, nitrophenols and nitrophenol derivatives are used in agricultural production as insecticides, fungicides, acaricides and herbicides.

There are numerous scientific researches and publications concerning the determination of the existence of compounds with organophosphates and carbamate through enzymatic methods.

One of this publications concerning the use of electrochemical biosensors for pesticide analysis that can be given as an example was written by Elif Burcu BAHADIR and Süreyya Meriç PAGANO in the Ni\check{g}de University Engineering Scientific Journal, Volume 3, Number 2, (2014), pages 18-28.

The aforementioned publication mentions that other than biosensors consisting of enzyme covered electrodes used in pesticide residue analysis, microbial organisms can be used as a biocatalyst element.

In these sensors, live organisms (algae, bacteria, yeast and fungus) can be selected as biocatalyst elements and the entire cell or part of it may be used for the biosensor. These microbial sensors are simple and cheap for some procedures, they don't require the elimination and purification of the enzymes and supply the necessary cofactor for the enzyme.

The biosensor in the amperometric microbial biosensors developed for the direct determination of p-nitrophenyl substitute organophosphate consists of the destructive agent p-nitrophenol. Pseudomonas putida JS444, immobilizes the organophosphor hydrolase enzyme on the cell surface of the immobilized carbon paste electrode and the electro oxidation flow is measured.

A bio-enzymatic conductometric biosensor has been developed with the Chlorella vulgaris as the bio-receptor. The algae is stored in the bovine serum albumin (BSA) with glutaraldehyde and is joined with the conductometric electrode. Local conductivity changes occur with the activities of the alg alcaline phosphatase and asetylcholine esterase.

Another example of biosensors for the determination of organophosphorus pesticides is the carbon pasta electrode prepared with genetically engineered cells of the organophosphor hydrolase indicated on the cell surface, paraokson, parathion and parathion methyl are hydrolysed to the p-nitrophenol. Determinations are made on the oxidisation flow measured anodically on the carbon transducer. If kept in 40 C for 45 days in perfect storage conditions it has 100% original activation.

The biosensor is prepared by way of storing Eschrerichia coli cultures on a policarbonate membrane and membrane is secured to the glass electrode with an O-ring. Responses to paraokson, parathion, parathion methyl and diazinon have been observed.

When considering field conditions, the fact the enzymatic biosensor retains 100% original activation when kept at 40 C for 45 days, is not a desirable situation. In addition, the pesticide active group in which the enzyme activates is limited to organopestide active materials.

In the aforementioned publication, DNA based biosensors are also mentioned. DNA biosensors based on guanine oxidisation have been used for pesticide analysis. These sensors are prepared with the DNA molecule fusing with various compounds and by way of the addition of electro-active analyte on the DNA redox properties or on the DNA plate. Voltammetry and potentiometry are commonly used electrochemical techniques.

Double strand calf thymus deoxyribonucleic acid attached to a polypyrrole polyvinyl sulphonate (ds-CT-DNA-Py-PVS) film is fabricated on indium tin oxide (ITO) covered glass plates. With this biosensor, chlorpyrifos has been determined as 0.0016-0.025 ppm and malation as 0.17-5 ppm.

Polianaline polyvinyl suphonate based DNA biosensors are fabricated using indium tin oxide (ITO) by way of the electrochemical attachment technique. The biosensors in which dsCT-DNA is attached to PANI-PVS/ITO by way of bio-electrode have determined the chlorpyrifos and malation as 0.5 ppb and 0.01 ppb accordingly. The response time is 30 s and the biosensor is stable for 6 months.

The procedures explained in the aforementioned publication have been carried out with enzyme based biosensors. A bio-receptor such as an enzyme unique to the substance that is to be analysed is required for the biosensor to be prepared. Whether the biosensor is enzyme based or cell based it fundamentally requires an enzyme.

The analyses of the samples given in the research have been conducted in laboratory conditions with the optimum conditions for the activation of the enzymes. In the debate section of the research, in the research of Cesarino et al. in 2012 on cabbages, broccoli and apples, carbaryl and metamyl pesticides were detected by use of a electrochemical acetylcholine biosensor and a HPLC/DAD (High Performance Liquid Chromatography/Diod Array Ddector) device. The cabbages, broccoli and apples used for the analysis were subjected to preliminary processes in laboratory conditions as required by the HPLC/DAD device properties.

The analysis kits mentioned in this research are enzyme and DNA based biosensors. They have been developed to facilitate the detection of pollutants in aquatic conditions. Sapling procedures were carried out with compounds containing organophosphate and carbamate. Carrying out analysis with biosensors in situ requires additional equipment and in this research, no information was given as to how to carry out pesticide analysis with a biosensor on plants, therefore the explanation pertains to the pesticide analyses prepared in laboratory conditions with samples having been subjected to preliminary processes. Samples collected from water can be directly entered into the biosensor with a drip cap and analysed; when dealing with water there is no protective barrier such as a fruit skin. Additional sampling set equipment is required for the juice of a fruit or vegetable with a skin to be extracted for analysis. In addition, the biological and physical properties of agriculturally cultivated plants differ from those of the surrounding water. The chemical structure of the plant sap and the compounds it contains are not the same as water.

As can be seen in the company products and scientific journal example mentioned above, not all of these compounds can be detected enzymatically and the determination of the amount cannot be carried out with the enzymatic test kits on the market. In addition, natural degeneration in the enzyme's chemical structure due on time observed in enzyme based kits degenerate the kit. Electrodes that are covered with compounds that do not contain DNA and for which the pesticide active material that is to cover the surface is not an enzyme are more sensitive than enzymatic kits; it is therefore possible to determine the existence of the pesticide, the chemical group, the active material belonging the chemical group and its quantity and pesticide metabolites and the lower limit for active material detection is the MRL value and can be reduced even further. The degeneration in the enzymatic kits mentioned above are not a concern for electrochemical kits that are covered with compounds that no not contain DNA and for which the surface is not covered with enzymes and they have a longer expected life.

The ambient temperature, pH value, ionic strength and other ambient conditions play an important role in enzyme based kits' enzymatic reactions. Negative conditions result in the enzyme losing its activity and the failure of the determination.

Microbial sensors used by living organisms also provide the necessary cofactor for the enzyme under optimum conditions; it is not always possible to ensure the desired optimum conditions in a field environment due to ambient factors and this shortens the expected life of the microbial sensor.

The procedure for the taking of the sample, the procedure for chemical solution application if necessary, the filtering, the precipitation of solids, the enzyme sensitive to the active material group for which the analysis is to be carried out, the living organism, the electrode with surface covered with surface material that doesn't contain DNA and DNA compounds are located in a single use set with predetermined ambient pressure, vacuum and gas content.

### SUMMARY OF THE INVENTION

One purpose of the invention is to realisation of a single use pesticide residue analysis kit set that includes, the sampling of the plant sap for the purpose of pesticide residue analysis before the harvest, the filtration, the application of a chemical solvent, the precipitation of solids, the application of the sample to an electrode to which surface covering material such as graphene that doesn't contain enzymes, living organism, DNA and DNA components, the connection to the field type pesticide residue analysis device for the purpose of reading of the ensuing chemical reaction and transforming the reading into data, in one single set.

A further purpose of the invention is to practically, and rapidly detect and determine pesticide residue and pesticide metabolites in the field with a kit set that does not include enzymes, living organisms, DNA and DNA components and to apply the non-enzymatic analysis method/methods to separate the pesticide active materials in the group.

### BRIEF DESCRIPTION OF THE DRAWINGS

FIG. 1 is a schematic view of one preferred embodiment of the pesticide analysis kit.

### DETAILED DESCRIPTION OF THE INVENTION

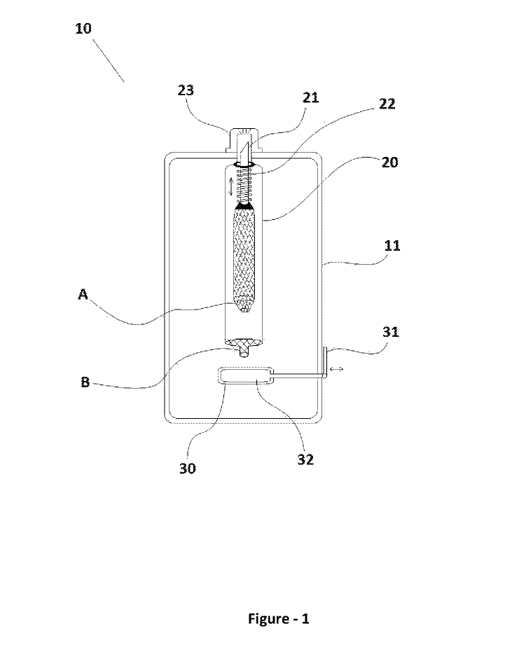

As shown in FIG. 1, The single use non-enzymatic residue analysis kit set 10 for non-enzymatic residue analysis in a field type pesticide analysis kit set 10 is made of transparent durable material and consists of an outer body in the shape of a capsule 11 that can be coloured depending on the pesticide active group that is to be analysed; the suction pressure for piercing and siphoning the plant sap can be adjusted, an injector 20 with springs 22 and vacuum; a capped needle 21 on the end of the injector 20 of which the diameter and length can be adjusted; the diameter of the holes can be adjusted in accordance with the properties of the plant sap that is to be analysed and for the purpose of filtering before the sample is applied to the electrode 30 to purify the plant sap that is sucked into the injector 20 of impurities and a polymerised filter A, B; a polymerised electrode 30 covered with graphene and/or other surface 32 covering material to ensure a reaction with pesticide active material ions that are to be determined, that does not include non-enzymatic, living organisms, DNA or DNA components depending on the pesticide active material group to be scanned and that has a chemical solution applied for the purpose of distancing unwanted molecules other than the molecules that are to be scanned in the filtered analysis material and drawing the desired molecules closer. The analysis surface 32 of the electrode 30 is inside the capsule 11 and the device connection points are on the outer surface 32 and are foldable.

The vacuum applied by the springed injector 20 when extracting sap from the plant for a residue analysis sample is sufficient for the sample to be sucked into the syringe, for the sample to pass through the filter A, B and for it to come into contact with the surface 32 of the electrode 30. The needle 21 length and the inner diameter of the hole can be customised according to the group of plants so as to penetrate the skin of the plant that is to be analysed and to access the sap. A gas, customised according to the pesticide active material group that is the be scanned is located in the capsule 11 to protect the properties during the pesticide active materiel group analysis process.

The electrochemical reaction that ensues on the electrode 30 in the capsule 11 is read once it is connected to the capsule 11 analysis device and transformed into data.

In the preferred application of the invention, the process of sample taking and analysis will be carried out by the non-enzymatic kit 10 that does not contain live organisms, DNA and DNA components in the capsule 11 shaped single set without having to relocate to a laboratory due to agricultural field conditions.

With the analysis kit 10 being located in the capsule 11, it is intended that the taking and preparation of the analysis sample be carried out immediately without needing to relocate to a laboratory.

With the analysis kit 10 being prepared without enzymes, living organisms, DNA or DNA compounds, in other words non-enzymatic, the analysis is intended that to be carried out rapidly and electrochemically with surfaces 32 covered with graphene or similar material and/or with polymerised electrodes 30.

With the outer part of the capsule 11 being sturdy, it is intended that the parts in the capsule 11 be protected in field conditions; with the capsule 11 being transparent, it is intended for the processes are observable and controllable; with the capsule 11 being colourful, it is intended that the analysis kits 10 can be distinguished depending on the different pesticide active materials groups that are to be analysed and ensure ease of use.

With the capsule 11 having a capped needle 21, it is intended that the injector 20 is sterile and it is possible to draw the plant sap into the capsule 11 by way of the vacuum.

With the capsule 11 containing a springed and vacuumed injector 20, it is intended that the amount of plant sap that is to be drawn for analysis is standardised.

With the inclusion of a polymerised filter A, B for which the diameter of the holes can be adjusted, it is intended that any materials other than the pesticide active materials in the plant sap are never analysed.

With the inclusion of an electrode 30 covered with graphene and/or similar surface 32 covering material that is to react with the pesticide active material ions that are to be determined and/or with a polymerised electrode 30, it is intended that the specified pesticide active material group reacts with the non-enzymatic, non-microbial material that does not contain DNA or DNA composites, that the graphene reduces the time required for the analysis with its reaction quickening effect; with the use of different surface 32 covering materials, it is intended that the intended chemical reaction is achieved by way of the ambiance selective property of the pesticide active material ions; with the application of chemical solutions to the electrode 30, it is intended that the reacting ions dissolve into the environment; with the electrode 30 being polymerised, it is intended that the intended reaction sensitivity be increased and that the unwanted ions are thrust away by the polymer from the electrode 30 and the desirable ions are drawn to the electrode 30.

With the analysis electrode end 31 being outside the capsule 11, it is intended that it be possible for the reaction in the capsule 11 to be read and transformed into data by the field type pesticide residue analysis device.

With the ends of the electrode 31 that is in the capsule 11 being outside the capsule 11, it is intended that the capsule 11 is easy to package and is protected from outside physical factors that could damage the delicate electrode ends 31.

With the inclusion of gas, the type of which is to be determined by the pesticide active material group, it is intended that the ions belonging to the pesticide active material group in the plant sap that is to be analysed retains its properties.

With the capsule 11 being a single use capsule 11, it is intended that the analysis is conducted in sterile conditions and that contamination is prevented.

### APPLICATION OF THE INVENTION IN THE INDUSTRY

The single use, non-enzymatic analysis kit set that does not contain living organisms, DNA or DNA compounds for field type pesticide residue analysis devices for the above mentioned purposes can be produced and used in any branch of the industry and is applicable in the industry.

### LIST OF REFERENCE NUMERALS

10. analysis kit
11. capsule
20. injector
21. needle
22. spring
23. lid
30. electrode
31. ends of electrode
32. surface
A. filter
B. filter

What is claimed is:

1. Land-type pesticide residue analysis device for disposable non-enzymatic, live micro-organisms, DNA does not contain sample analysis the components of the kit and set 10 whether the property; durable and made of transparent material and colour can be changed in case of an external body of the capsule 11 to retrieve the capsule 11 plant sample drilling and plant to take into its SAP, the pressure can be adjusted, spring 22 and vacuum injector 20; it's on the tip of the syringe's diameter and length adjustable slider pin yourself; foreign molecules in the plant SAP taken filler to purify the sample electrode 30 for the purposes of filtering and analysis before will be made according to the properties of the plant SAP can vary in diameter of hole polymerised filter A, B; in the scan to be done analyzing the filtered molecules away from the unwanted molecules outside the environment, closer to the desired molecules in the environment have been treated with a chemical solution and polymerised to be scanned if the pesticide active ingredient group over non-enzymatic, live micro-organisms, DNA does not contain any pesticide active substances and components, ion will react with Graphene to be elected on the property and/or other surface 32 coating material can be folded out of the capsule 11 and coated with property on the ends of the electrode 31; the capsule 11 will be determined according to the pesticide active ingredient group within the gas.

2. According to the claim 1, land-type pesticide residue analysis device for disposable non-enzymatic, live micro-organisms, DNA does not contain sample analysis the components of the kit, and whether the property; durable and made of transparent material and colour can be changed in case of an external body of the capsule 11.

3. According to the claim 2, land-type pesticide residue analysis device for disposable non-enzymatic, live micro-organisms, DNA does not contain sample analysis the components of the kit, and whether the property; to retrieve the capsule 11 plant sample drilling and plant to take into its SAP, the pressure can be adjusted, spring 22 and vacuum injector 20.

4. According to the claim 3, land-type pesticide residue analysis device for disposable non-enzymatic, live micro-organisms, DNA does not contain sample analysis the components of the kit and set 10 whether the property; the syringe tip diameter and length adjustable self is a needle 21 with a lid 23.

5. According to the claim 4, land-type pesticide residue analysis device for disposable non-enzymatic, live micro-organisms, DNA does not contain sample analysis the components of the kit and set 10 whether the property; foreign molecules in the plant SAP taken filler to purify the sample electrode 30 for the purposes of filtering and analysis before will be made according to the properties of the plant SAP can vary in diameter of holes and polymerised filter A, B.

6. According to the claim 5, pesticide residue analysis device for disposable non-enzymatic, live micro-organisms, DNA does not contain sample analysis the components of the kit and set 10 whether the property; in the scan to be done analyzing the filtered molecules away from the unwanted molecules outside the environment, closer to the desired molecules in the environment have been treated with a chemical solution and polymerised to be scanned if the pesticide active ingredient group over non-enzymatic, live micro-organisms, DNA and does not contain any components of Graphene and/or other surface 32 coating with coated, capsule 11 can be folded to the ends of the property other than the electrode 31.

7. According to the claim 6, pesticide residue analysis device for disposable non-enzymatic, live micro-organisms, DNA does not contain analysis of the components of the kit and set 10 whether the property; the capsule 11 will be determined according to the pesticide active ingredient group within the gas.

8. Non-enzymatic analysis of pesticides in field conditions in pesticide residue analysis device practically land type non-enzymatic, live micro-organisms, DNA does not contain components of surface 32 coated and polymerized and non-enzymatic, living organism, DNA and does not contain the components of the method that is to be done with the kit that contains electrodes 30 property:
a. Sample the SAP of the automatic filler Kit sterile;
b. Injector 20 vacuum dint polymerized filtering A, B;
c. Live microorganisms, enzymes, DNA and bio-catalytic element that contains unused components of Graphene and/or covered with other surface 32 coating material and/or polymerized electrodes 30 should be treated; and
d. An analysis of the reaction that occurs in a handheld device can be seen in the pesticide by reading numeric data is becoming.
